SEQUENCE LISTING (10 sequences)

SEQ ID NO: 1 - DNA, 35 bp, Artificial sequence
  Synthetic oligonucleotide
gcgcagatctatgacaaacctgtcagatcaaaccc

SEQ ID NO: 2 - DNA, 34 bp, Artificial sequence
  Synthetic oligonucleotide
gcgcaagcttaggcgagagtcagctgccttatgc

SEQ ID NO: 3 - DNA, 7595 bp, Artificial sequence
  Synthetic plasmid
gggtgatcaagtcttcgtcgagtgattgtaaataaaatgtaatttacagtatagtattttaattaatatacaaatgatttgataataattcttatttaactataatatattgtgttgggttgaattaaaggtccgtatactccggaatattaatagatcatggagataattaaaatgataaccatctcgcaaataaataagtattttactgttttcgtaacagttttgtaataaaaaaacctataaatattccggattattcataccgtcccaccatcgggcgcggatctatgacaaacctgtcagatcaaacccagcagattgttccgttcatacggagccttctgatgccaacaaccggaccggcgtccattccggacgacaccctggagaagcacactctcaggtcagagacctcgacctacaatttgactgtgggggacacagggtcagggctaattgtctttttccctggattccctggctcaattgtgggtgctcactacacactgcagggcaatgggaactacaagttcgatcagatgctcctgactgcccagaacctaccggccagttacaactactgcaggctagtgagtcggagtctcacagtgaggtcaagcacacttcctggtggcgtttatgcactaaacggcaccataaacgccgtgaccttccaaggaagcctgagtgaactgacagatgttagctacaatgggttgatgtctgcaacagccaacatcaacgacaaaattgggaacgtcctagtaggggaaggggtcaccgtcctcagcttacccacatcatatgatcttgggtatgtgaggcttggtgaccccattcccgcaatagggcttgacccaaaaatggtagccacatgtgacagcagtgacaggcccagagtctacaccataactgcagccgatgattaccaattctcatcacagtaccaaccaggtggggtaacaatcacactgttctcagccaacattgatgccatcacaagcctcagcgttgggggagagctcgtgtttcgaacaagcgtccacggccttgtactgggcgccaccatctacctcataggctttgatgggacaacggtaatcaccagggctgtggccgcaaacaatgggctgacgaccggcaccgacaaccttatgccattcaatcttgtgattccaacaaacgagataacccagccaatcacatccatcaaactggagatagtgacctccaaaagtggtggtcaggcaggggatcagatgtcatggtcggcaagagggagcctagcagtgacgatccatggtggcaactatccaggggccctccgtcccgtcacgctagtggcctacgaaagagtggcaacaggatccgtcgttacggtcgctggggtgagcaacttcgagctgatcccaaatcctgaactagcaaagaacctggttacagaatacggccgatttgacccaggagccatgaactacacaaaattgatactgagtgagagggaccgtcttggcatcaagaccgtctggccaacaagggagtacactgactttcgtgaatacttcatggaggtggccgacctcaactctcccctgaagattgcaggagcattcggcttcaaagacataatccgggccataaggaggatagctgtgccggtggtctccacattgttcccacctgccgctcccctagcccatgcaattggggaaggtgtagactacctgctgggcgatgaggcccaggccgcttcaggaactgctcgagccgcgtcaggaaaagcaagagctgcctcaggccgcataaggcagctgactctcgcctaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggcctacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgcctcgagactgcaggctctagattcgaaagcggccgcgactagtgagctcgtcgacgtaggcctttgaattccggatcctcactcaaggtcctcatcagagacggtcctgatccagcggcccagccgaccagggggtctctgtgttggagcattgggttttggcttgggctttggtagagcccgcctgggattgcgatgcttcatctccatcgcagtcaagagcagatctttcatctgttcttggtttgggccacgtccatggttgatttcatagactttggcaacttcgtctatgaaagcttggggtggctctgcctgtcctggagccccgtagatcgacgtagctgcccttaggatttgttcttctgatgccaaccggctcttctctgcatgcacgtagtctagatagtcctcgtttgggtccggtatttctcgtttgttctgccagtactttacctggcctgggcttggccctcggtgcccattgagtgctacccattctggtgttgcaaagtagatgcccatggtctccatcttctttgagatccgtgtgtctttttccctctgtgcttcctctggtgtggggccccgagcctccactccgtagcctgctgtcccgtacttggccctttgcgacttgctgcctgcttgtggtgcgtttgcaagaaaatttcgcatccgatgggcgttcgggtcgctgagtgcgaagttggccatgtcagtcacaatcccattctcttccagccacatgaacacactgagtgcagattggaatagtgggtccacgttggctgctgcttccattgctctgacggcactctcgagttcgggggtctctttgaactctgatgcagccatggcgccctgaaaatacaggttttcggtcgttgggatatcgtaatcgtgatggtgatggtgatggtagtacgacatggtttcggac

SEQ ID NO: 4 - protein, 333 aa, Artificial sequence
  Synthetic polypeptide
MTNLSDQTQQIVPFIRSLLMPTTGPASIPDDTLEKHTLRSETSTYNLTVGDTGSGLIVFFPGFPGSIVGAHYTLQGNGNYKFDQMLLTAQNLPASYNYCRLVSRSLTVRSSTLPGGVYALNGTINAVTFQGSLSELTDVSYNGLMSATANINDKIGNVLVGEGVTVLSLPTSYDLGYVRLGDPIPAIGLDPKMVATCDSSDRPRVYTITAADDYQFSSQYQPGGVTITLFSANIDAITSLSVGGELVFRTSVHGLVLGATIYLIGFDGTTVITRAVAANNGLTTGTDNLMPFNLVIPTNEITQPITSIKLEIVTSKSGGQAGDQMSWSARGSL

SEQ ID NO: 5 - DNA, 35 bp, Artificial sequence; identical to SEQ ID NO: 1
  Synthetic oligonucleotide

SEQ ID NO: 6 - DNA, 34 bp, Artificial sequence; identical to SEQ ID NO: 2
  Synthetic oligonucleotide

SEQ ID NO: 7 - DNA, 33 bp, Artificial sequence
  Synthetic oligonucleotide
gcgcgaattcgatggcatcagagttcaaagaga

SEQ ID NO: 8 - DNA, 32 bp, Artificial sequence
  Synthetic oligonucleotide
cgcggatccctcaaggtcctcatcagagacgg

SEQ ID NO: 9 - DNA, 9600 bp, Artificial sequence
  Synthetic plasmid
ggccgcactagtatcgatggattacaaggatgacgacgataagatctgagctcttaattaacaattcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctccaattcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggatcttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatcttagctagccgcggtaccaagcttaggcgagagtcagctgccttatgcggcctgaggcagctcttgcttttcctgacgcggctcgagcagttcctgaagcggcctgggcctcatcgcccagcaggtagtctacaccttccccaattgcatgggctaggggagcggcaggtgggaacaatgtggagaccaccggcacagctatcctccttatggcccggattatgtctttgaagccgaatgctcctgcaatcttcaggggagagttgaggtcggccacctccatgaagtattcacgaaagtcagtgtactcccttgttggccagacggtcttgatgccaagacggtccctctcactcagtatcaattttgtgtagttcatggctcctgggtcaaatcggccgtattctgtaaccaggttctttgctagttcaggatttgggatcagctcgaagttgctcaccccagcgaccgtaacgacggatcctgttgccactctttcgtaggccactagcgtgacgggacggagggcccctggatagttgccaccatggatcgtcactgctaggctccctcttgccgaccatgacatctgatcccctgcctgaccaccacttttggaggtcactatctccagtttgatggatgtgattggctgggttatctcgtttgttggaatcacaagattgaatggcataaggttgtcggtgccggtcgtcagcccattgtttgcggccacagccctggtgattaccgttgtcccatcaaagcctatgaggtagatggtggcgcccagtacaaggccgtggacgcttgttcgaaacacgagctctcccccaacgctgaggcttgtgatggcatcaatgttggctgagaacagtgtgattgttaccccacctggttggtactgtgatgagaattggtaatcatcggctgcagttatggtgtagactctgggcctgtcactgctgtcacatgtggctaccatttttgggtcaagccctattgcgggaatggggtcaccaagcctcacatacccaagatcatatgatgtgggtaagctgaggacggtgaccccttcccctactaggacgttcccaattttgtcgttgatgttggctgttgcagacatcaacccattgtagctaacatctgtcagttcactcaggcttccttggaaggtcacggcgtttatggtgccgtttagtgcataaacgccaccaggaagtgtgcttgacctcactgtgagactccgactcactagcctgcagtagttgtaactggccggtaggttctgggcagtcaggagcatctgatcgaacttgtagttcccattgccctgcagtgtgtagtgagcacccacaattgagccagggaatccagggaaaaagacaattagccctgaccctgtgtcccccacagtcaaattgtaggtcgaggtctctgacctgagagtgtgcttctccagggtgtcgtccggaatggacgccggtccggttgttggcatcagaaggctccgtatgaacggaacaatctgctgggtttgatctgacaggtttgtcatagatccggggttttttctccttgacgttaaagtatagaggtatattaacaattttttgttgatacttttattacatttgaataagaagtaatacaaaccgaaaatgttgaaagtattagttaaagtggttatgcagtttttgcatttatatatctgttaatagatcaaaaatcatcgcttcgctgattaattaccccagaaataaggctaaaaaactaatcgcattatcatcctatggttgttaatttgattcgttcatttgaaggtttgtggggccaggttactgccaatttttcctcttcataaccataaaagctagtattgtagaatctttattgttcggagcagtgcggcgcgaggcacatctgcgtttcaggaacgcgaccggtgaagacgaggacgcacggaggagagtcttccttcggagggctgtcacccgctcggcggcttctaatccgtacttcaatatagcaatgagcagttaagcgtattactgaaagttccaaagagaaggtttttttaggctaagataatggggctctttacatttccacaacatataagtaagattagatatggatatgtatatggatatgtatatggtggtaatgccatgtaatatgattattaaacttctttgcgtccatccaaaaaaaaagtaagaatttttgaaaattcgaattcgatggctgcatcagagttcaaagagacccccgaactcgagagtgccgtcagagcaatggaagcagcagccaacgtggacccactattccaatctgcactcagtgtgttcatgtggctggaagagaatgggattgtgactgacatggccaacttcgcactcagcgacccgaacgcccatcggatgcgaaattttcttgcaaacgcaccacaagcaggcagcaagtcgcaaagggccaagtacgggacagcaggctacggagtggaggctcggggccccacaccagaggaagcacagagggaaaaagacacacggatctcaaagaagatggagaccatgggcatctactttgcaacaccagaatgggtagcactcaatgggcaccgagggccaagcccaggccaggtaaagtactggcagaacaaacgagaaataccggacccaaacgaggactatctagactacgtgcatgcagagaagagccggttggcatcagaagaacaaatcctaagggcagctacgtcgatctacggggctccaggacaggcagagccaccccaagctttcatagacgaagttgccaaagtctatgaaatcaaccatggacgtggcccaaaccaagaacagatgaaagatctgctcttgactgcgatggagatgaagcatcgcaatcccaggcgggctctaccaaagcccaagccaaaacccaatgctccaacacagagaccccctggtcggctgggccgctggatcaggaccgtctctgatgaggaccttgagggatccatcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagc

SEQ ID NO: 10 - protein, 503 aa, Artificial sequence
  Synthetic polypeptide
MAASEFKETPELESAVRAMEAAANVDPLFQSALSVFMWLEENGIVTDMANFALSDPNAHRMRNFLANAPQAGSKSQRAKYGTAGYGVEARGPTPEEAQREKDTRISKKMETMGIYFATPEWVALNGHRGPSPGQVKYWQNKREIPDPNEDYLDYVHAEKSRLASEEQILRAATSIYGAPGQAEPPQAFIDEVAKVYEINHGRGPNQEQMKDLLLTAMEMKHRNPRRALPKPKPKPNAPTQRPPGRLGRWIRTVSDEDLEGSIATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

(19) United States
(12) Patent Application Publication
(10) Pub. No.: US 2009/0208528 A1
(43) Pub. Date: Aug. 20, 2009

(54) Empty capsids (vlps(-vp4)) of the infectious bursal disease virus (ibdv), obtainment process and applications

(76) Inventors: Jose Francisco Rodriguez Aguirre, Madrid (ES); Jose Ruiz Caston, Madrid (ES); Maria Dolores Gonzalez de Llano, Madrid (ES); Ana Maria Ona Blanco, Madrid (ES); Fernando Abaitua Elustondo, Oxted (GB); Daniel Luque Buzo, Madrid (ES); Juan Ramon Rodriguez Fernandez-Alba, Madrid (ES)

Correspondence Address: KLARQUIST SPARKMAN, LLP, 121 SW SALMON STREET, SUITE 1600, PORTLAND, OR, 97204, US

(21) Appl. No.: 10/576,988

(22) PCT Filed: Jan. 21, 2005
(86) PCT No.: PCT/EP2005/000694

§ 371 Date: Oct. 8, 2008

(30) Foreign Application Priority Data
Jan. 21, 2004 (ES) P200400121

(51) Int. Cl. A61K 39/12; C12N 7/01; C12N 15/40; C12N 15/85; C12N 5/10; C12N 1/19; C12P 21/02; C12N 15/81; C07K 14/08; A61P 31/14

(52) U.S. Cl. 424/204.1; 435/235.1; 536/23.72; 435/320.1; 435/348; 435/254.2; 435/69.1; 530/350

(57) ABSTRACT

The empty capsids of the infectious bursal disease virus (IBDV), VLP(−VP4), are characterized in that they are constituted only by assembly of IBDV pVP2 proteins and IBDV VP3 proteins. Said capsids have immunogenic activity and can be used in the manufacture of vaccines for protecting animals from the infection caused by IBDV, as well as in the manufacture of gene therapy vectors.

### FIELD OF THE INVENTION

The invention is related to empty viral capsids of the infectious bursal disease virus (IBDV), with immunogenic activity against IBDV, constituted by the IBDV VP3 and pVP2 proteins, to the production thereof by means of genetic engineering, and to their applications, particularly in the manufacture of vaccines against the avian disease called infectious bursal disease caused by IBDV, and in the manufacture of gene therapy vectors.

### BACKGROUND OF THE INVENTION

The infectious bursal disease virus (IBDV), also known as Gumboro disease, belongs to the Birnaviridae family, infects different bird species and is directly responsible for a severe immunosuppressive disease causing important economic losses in the world poultry industry.

IBDV particles are icosahedral, with T=13 symmetry, they lack an envelope and are formed by a single protein layer. Up until now, the approaches aimed at obtaining an atomic model for IBDV particles have failed. As a result, the structural information available is based on three-dimensional models generated from images obtained by electron cryomicroscopy of the purified virus and of the VLPs. Based on these studies, it has been verified that the outer surface of the particle is formed by a continuous lattice of 260 trimers of the VP2 protein (37 kDa) organized in five different formations. The inner face of the particles contains 200 trimers of the VP3 protein (29 kDa), the latter, independent from one another, are bound to the basal area of the VP2 trimers. It has been suggested that a third polypeptide, VP4 (28 kDa), could also be part of the particles, being located at the base of the pentamers forming the vertices of the icosahedral structure.

The VP2, VP3 and VP4 polypeptides are produced from the proteolytic processing of a polypeptide precursor of a size of 109 kDa. This precursor is auto-catalytically processed, releasing the pVP2 (48 kDa), VP3 and VP4 polypeptides. The VP4 domain, which is located in the central region of the polyprotein, belongs to the Lon protease family and is responsible for the proteolytic cleavage. The pVP2 and VP3 polypeptides are directly responsible for the capsid assembly. The pVP2 product undergoes a last cleavage at its C-terminal end before giving rise to the mature form of the protein, VP2, which is the one found in purified particles (Da Costa, B., Chevalier, C., Henry, C., Huet, J. C., Petit, S., Lepault, J., Boot, H. & Delmas, B. (2002). The capsid of infectious bursal disease virus contains several small peptides arising from the maturation process of pVP2. Journal of Virology 76:2393-2402). This pVP2 processing is necessary for the correct formation of the capsids and requires the presence of VP3, although the responsible protease has not yet been identified (Maraver, A., Oña, A., Abaitua, F., González, D., Clemente, R., Diaz-Ruiz, A., Caston, J. R., Pazos, F. & Rodríguez, J. F. (2003). The oligomerization domain of VP3, the scaffolding protein of infectious bursal disease virus, plays a critical role for capsid formation. Journal of Virology 77:6438-49).

Conventional vaccines used for the control of infectious bursal disease are based on the use of strains, with different degrees of virulence, of IBDV itself grown in cell culture or in embryonated eggs. The extracts containing the infectious material are subjected to chemical inactivation processes to produce inactivated vaccines, or else are used directly to produce live attenuated vaccines (Sharma, J. M., Kim, I. J., Rautenschlein, S. & Yeh, H. Y. (2000). Infectious bursal disease virus of chickens: pathogenesis and immunosuppression. Developmental and Comparative Immunology 24:223-235; van den Berg T P, Eterradossi N, Toquin D, Meulemans G. 2000. Rev Sci Tech 2000, 19:509-543). This latter type of vaccines has the typical drawbacks associated with the use of live attenuated vaccines, specifically, the risk of mutations reverting the virulence of the virus or making it lose its immunogenicity.

Recombinant subunit vaccines containing the IBDV protein VP2 expressed in several expression systems, for example, bacteria, yeasts or baculovirus, usually in fusion protein form, have been disclosed. The results obtained in chicken immunization tests with said vaccines have not been completely satisfactory.

Empty viral capsids or virus-like particles (VLPs) constitute an alternative to the use of live attenuated vaccines and of recombinant subunit vaccines. VLPs are obtained by self-assembly of the subunits constituting the viral capsid and mimicking the structure and antigenic properties of the native virion, even though they lack genetic material, as a result of which they are incapable of replicating themselves. Apart from their application for vaccination purposes, VLPs can be used as vectors of molecules of biological interest, for example, nucleic acids, peptides or proteins. By way of illustration, parvovirus VLPs (U.S. Pat. No. 6,458,362) or human immunodeficiency syndrome (HIV) VLPs (U.S. Pat. No. 6,602,705), can be mentioned.

Morphogenesis is a vital process for the viral cycle requiring successive steps associated to modifications in the polypeptide precursors. As a result, viruses have developed strategies allowing the sequential and correct interaction between each one of their components. One of these strategies, frequently used by icosahedral viruses, is the use of polypeptides coming from a single polyprotein as the base of their structural components. In these cases, the suitable proteolytic processing of said polyprotein plays a crucial role in the assembly process.

This concept for the assembly of IBDV capsids has been demonstrated in earlier work (Fernández-Arias, A., Risco, C., Martínez, S., Albar, J. P. & Rodríguez, J. F. (1998). Expression of ORF A1 of infectious bursal disease virus results in the formation of virus-like particles. Journal of General Virology 79, 1047-1054). Expression of the gene encoding for the IBDV polyprotein in eukaryotic cells gives rise to the formation of VLPs that are completely morphologically and biochemically indistinguishable from the IBDV virions. It has also been shown that the assembly of the capsids requires only the synthesis and correct processing of the viral polyprotein and is independent of the presence of the viral genome or of other proteins encoded by the viral genome, such as VP5 and VP1.

Parallelly to the formation of capsids, the IBDV VP4 product is able to self-assemble in tubular structures of 20 nm in diameter. These tubules, known as type II tubules, are partially copurified with the viral particles. Other experiments have demonstrated that obtaining IBDV VLPs, using recombinant baculoviruses (rBVs) for polyprotein expression, is extremely inefficient, the accumulation of large amounts of type I tubules in the cytosol of the infected cells being obtained (Martínez-Torrecuadrada, J. L., Castón, J. R., Castro, M., Carrascosa, J. L., Rodríguez, J. F. & Casal, J. I. (2000). Different architectures in the assembly of infectious bursal disease virus capsid proteins expressed in insect cells. Virology 278:322-331; Chevalier, C., Lepault, J., Erk, I., Da Costa, B. & Delmas, B. (2002). The maturation process of pVP2 requires assembly of infectious bursal disease virus capsids. Journal of Virology 76:2384-2392). It has recently been demonstrated that this is due to the proteolytic cleavage to which the VP3 protein is subjected when it is synthesized in insect cells. Said proteolysis is almost completely prevented by the formation of VP3/VP1 complexes (Maraver, A., Oña, A., Abaitua, F., González, D., Clemente, R., Diaz-Ruiz, A., Caston, J. R., Pazos, F. & Rodríguez, J. F. (2003). The oligomerization domain of VP3, the scaffolding protein of infectious bursal disease virus, plays a critical role for capsid formation. Journal of Virology 77:6438-6449).

Therefore, the results obtained to date from the IBDV gene expression in different recombinant systems has allowed concluding that: (i) the assembly process is independent of the presence of genetic material of the virus, (ii) only the polypeptides encoded by the polyprotein gene are necessary for the assembly, and (iii) the assembly requires a coordinated interaction between the pVP2 and VP3 peptides.

However, it is not known if the VP2/VP3 interaction is established between VP2 and VP3 domains of the polyprotein precursor when it has not yet undergone modifications, or on the contrary, if this interaction occurs after the processing of the precursor. Furthermore, current information does not exclude the possibility that VP4 could play a relevant role in the morphogenesis of the viral capsid. In fact, IBDV VLPs formed by assembly of the IBDV VP2, VP3 and VP4 proteins have been disclosed (U.S. Pat. No. 6,528,063, U.S. Pat. No. 5,788,970 and JP 5194597).

On the other hand, information regarding IBDV protein expression by means of genetic engineering in different cell models is scarce. IBDV protein expression in insect cells, bacteria and yeasts has been disclosed. Jagadish et al. (Jagadish M N, Vaughan P, Irving R A, Azad A A, Macreadie I G. (1990). Expression and characterization of infectious bursal disease virus polyprotein in yeast. Gene 9:179-186; Macreadie I G, Vaughan P R, Chapman A J, McKern N M, Jagadish M N, Heine H G, Ward C W, Fahey K J, Azad A A. (1990). Passive protection against infectious bursal disease virus by viral VP2 expressed in yeast. Vaccine 8:549-552) disclose IBDV VP2 expression in yeasts. The disclosed results indicate that the viral polyprotein expression in two yeast species, Saccharomyces cerevisiae and Saccharomyces pombe, is very inefficient, a large variety of protein products of different molecular mass being accumulated. The failure to obtain protein products of the expected size and the inability to detect structures produced due to the assembly of the latter was attributed to two possible causes: (i) possible toxicity of the IBDV protease (VP4) in this system; and/or (ii) the inefficiency of the expression system to carry out a correct transcription and/or translation of the IBDV polyprotein. Recently, Pitcovski et al. (Pitcovski J., Gutter B, Gallili G, Goldway M, Perelman B, Gross G, Krispel S, Barbakov M, Michael A. (2003). Vaccine 21:4736-4743) have disclosed the IBDV VP2 expression in Pichia pastoris and the immunization of chickens with a material comprising the recombinant protein (rVP2) in partially purified form. In no case has the obtainment of IBDV VLPs in yeast been disclosed.

Earlier work developed by the inventors has enabled establishing systems for obtaining IBDV VLPs using different eukaryotic expression vectors. These vectors have been used for IBDV polyprotein expression in the absence or presence of the viral VP1 RNA polymerase. The biochemical characterization of purified VLPs demonstrates that they contain pVP2, VP2 and VP3 proteins when only the viral polyprotein is expressed, and the pVP2, VP2, VP3 and VP1 proteins when the simultaneous expression of the polyprotein and viral RNA polymerase is carried out (Fernández-Arias, A., Risco, C., Martínez, S., Albar, J. P. & Rodríguez, J. F. (1998). Expression of ORF A1 of infectious bursal disease virus results in the formation of virus-like particles. Journal of General Virology 79: 1047-1054; Martínez-Torrecuadrada, J. L., Castón, J. R., Castro, M., Carrascosa, J. L., Rodríguez, J. F. & Casal, J. I. (2000). Different architectures in the assembly of infectious bursal disease virus capsid proteins expressed in insect cells. Virology 278: 322-331; Maraver, A., et al., (2003) cited supra; Lombardo, E., Maraver, A., Castón, J. R., Rivera, J., Fernández-Arias, A., Serrano, A., Carrascosa, J. L. & Rodríguez, J. F. (1999). VP1, the putative RNA-dependent RNA polymerase of infectious bursal disease virus, forms complexes with the capsid protein VP3, leading to efficient encapsidation into virus-like particles. Journal of Virology 73: 6973-6983). However, VLPs solely based on IBDV pVP2 and VP3, or their potential use for vaccine purposes or as vehicles of biological products of interest, have not been previously disclosed.

### SUMMARY OF THE INVENTION

The invention is faced with the problem of providing new effective and safe vaccines against the infectious bursal disease virus (IBDV).

The solution provided by this invention is based on it being possible to obtain correctly assembled IBDV VLPs by means of the simultaneous expression of the IBDV pVP2 and VP3 polypeptides as independent genes and as the only representation of IBDV proteins in a gene expression system. Said VLPs are formed by self-assembly of only IBDV pVP2 and VP3, whereby they lack IBDV VP4 and, for this reason, are called VLP(−VP4) (singular) or VLPs(−VP4) (plural) in this description. Said VLPs(−VP4) can be used for example for therapeutic or diagnostic purposes, for example, in the manufacture of vaccines to protect birds from the infection caused by IBDV, or in the manufacture of gene therapy vectors.

The obtained results allow conceiving two new conclusions to the understanding of the IBDV assembly pattern: (i) the interactions between the pVP2/VP3 polypeptides result in an efficient assembly of the IBDV particles without needing expression of the whole polyprotein, and (ii) the VP4 polyprotein is not required for capsid formation.

These results have allowed designing a new strategy or process for the efficient production of IBDV VLPs containing antigenically relevant protein elements so as to induce an immune response. This strategy is based on the use of a gene expression system or vector allowing the coexpression of pVP2 and VP3 polypeptides as independent genes and preventing the synthesis of the polyprotein precursor of said polypeptides as well as the presence of the VP4 polypeptide during the empty viral capsid assembly process.

The expression and obtainment of IBDV VLPs, particularly VLPs(−VP4), in insect cells is described in a particular embodiment, while in another particular embodiment, said VLPs(−VP4) are obtained in yeasts, with a very high yield and a very low economic cost.

The vaccines obtained using said VLPs(−VP4) have a number of advantages since, on one hand, the handling of highly infectious material is prevented, the potential risk of the occurrence of new IBDV mutants is prevented and the use of a live virus in poultry farms is eliminated, thus preventing the risk of spreading IBDV vaccine strains to the environment, and on the other hand, it enables the development of differential diagnostic systems to discriminate between vaccinated and infected animals. These diagnostic systems are based on the detection of antibodies against VP2 and VP4 proteins. Animals with IBDV develop a strong humoral response to both proteins. However animals immunized with VLPs(−VP4) only have antibodies against the VP2 protein.

Consequently, an object of the present invention consists of an empty IBDV viral capsid, VLP(−VP4), with immunogenic activity against infection in IBDV, characterized in that it is constituted by self-assembly of only IBDV pVP2 and VP3 proteins.

A further aspect of this invention is related to a process for producing said IBDV VLPs(−VP4) provided by this invention, based on the gene coexpression of said IBDV pVP2 and VP3 proteins as two independent genes.

The nucleic acids, gene constructs, expression systems and host cells developed for implementing said process of producing said IBDV VLPs(−VP4), as well as their use for the production of said IBDV VLPs(−VP4), constitute further aspects of the present invention.

Said IBDV VLPs(−VP4) have the ability to immunize animals, particularly birds, against the avian disease caused by IBDV, as well as the ability to incorporate in vectors or vehicles molecules of biological interest, for example, polypeptides, proteins, nucleic acids, etc. In a particular embodiment, said IBDV VLPs(−VP4) can be used in the manufacture of a vaccine to protect birds against the virus causing the avian disease known as infectious bursal disease (IBDV). Virtually any bird, preferably those avian species of economic interest, for example, chickens, turkeys, geese, ganders, pheasants, quails, ostriches, etc., can be immunized against the infection caused by IBDV with the vaccines provided by this invention. In another particular embodiment, said IBDV VLPs(−VP4) can internally incorporate into vehicles products with biological activity, for example, nucleic acids, peptides, proteins, drugs, etc., whereby they can be used in the manufacture of gene therapy vectors.

Therefore, in a further aspect, the present invention is related to the use of said IBDV VLPs(−VP4), in the manufacture of medicaments, such as vaccines and gene therapy vectors. Said vaccines and vectors constitute further aspects of the present invention. In a particular embodiment, said vaccine is a vaccine useful for protecting birds from the infection caused by IBDV. In a specific embodiment, said birds are selected from the group formed by chickens, turkeys, geese, ganders, pheasants, quails and ostriches, preferably chickens.

### BRIEF DESCRIPTION OF THE FIGURES

FIG. 1. (a) The diagram schematizes the proteolytic processing steps necessary for the formation of mature VP2 and VP3 capsid proteins from the polyprotein precursor. (b) The diagram reflects the different genetic constructs derived from the IBDV polyprotein described up until now, as well as the structures produced by means of its expression in different heterologous systems. The numbers indicate the position corresponding to the first and last amino acid residue of the polyprotein present in each one of the constructs. The lower portion of the figure shows images obtained by means of transmission electron microscopy of the structures obtained by means of expression of the different constructs. The bar corresponds to 50 nm. The data has been taken from the following literature references: Fernández-Arias et al., (1998), cited supra; Maraver et al., (2003), cited supra; Martínez-Torrecuadrada et al., (2000), cited supra; Castón et al., 2001. C terminus of infectious bursal disease virus major capsid protein VP2 is involved in definition of the t number for capsid assembly. Journal of Virology 75, 10815-10828.

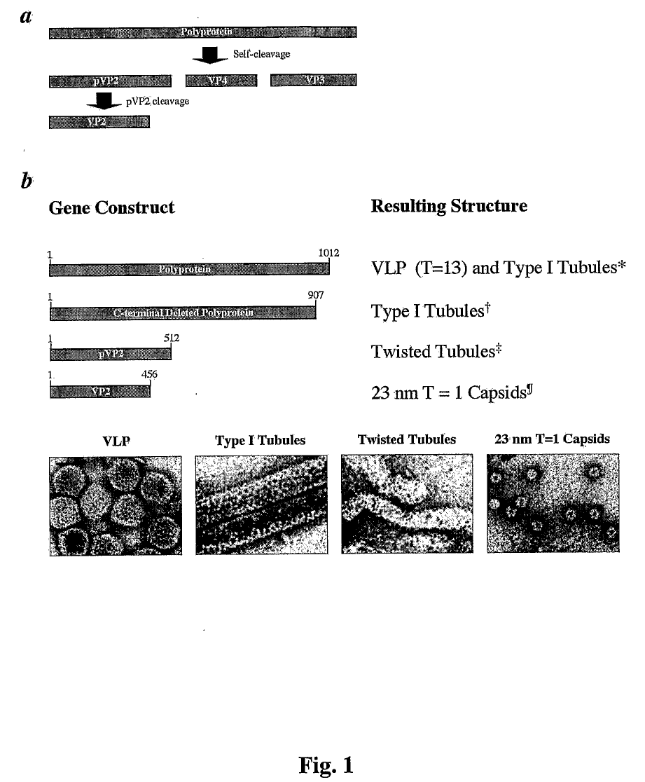

FIG. 2. Microscopic analysis of H5 insect cells coexpressing pVP2 and VP3. The pVP2 and VP3 protein subcellular distribution was analyzed by means of confocal immunomicroscopy. Cells infected with the FB/pVP2 (a), FB/VP3 (b), or FBD/pVP2-VP3 (c-e) rBVs were incubated with anti-pVP2 rabbit serum and anti-VP3 rat serum. Then the cells were incubated with goat anti-rabbit IgG serum coupled to Alexa 488 (red) and goat anti-rat IgG serum coupled to Alexa 594 (green). The cores were stained with To-Pro 3 (blue). (e) Overlaying of the images shown in panels (c) and (d). Electron microscopy images corresponding to sections of H5 cells infected with different genetic constructs derived from the IBDV polyprotein. (f) Low-magnification image of an H5 cell infected with a parental Fb virus. The insert corresponds to an enlarged detail of the area indicated by the box. (g) Low-magnification image of an H5 cell infected with the FBD/pVP2-VP3 virus. The insert corresponds to an enlarged detail of the area indicated by the box. (h) High-magnification image of an H5 cell infected with the FBD/pVP2-VP3 virus showing the formation of IBDV structures in detail. (i) High-magnification image of a BSC1 cell infected with the VTLacOI/POLY recombinant vaccine virus showing structures similar to those detected in panel (h). The bars indicate 600 nm (panels f and g) and 200 nm (panels h and i).

FIG. 3. Structural and biochemical characterization of the structures derived from IBDV produced in insect cells coinfected with the (rBV) FB/pVP2+FB/his-VP3 recombinant baculoviruses. Cells coinfected with FB/pVP2 and FB/his-VP3 rBVs, or infected with the FBD/Poly-VP1 or FB/pVP2 virus, were used to purify structures derived from IBDV by means of centrifugation on sucrose gradients. Panels (a), (b), and (c) show transmission electron microscopy images corresponding to fraction 4 of the gradients obtained from infections with FBD/Poly-VP1, FB/pVP2+FB/his-VP3, and FB/pVP2, respectively. Panel (d) shows the results of a Western blot analysis of the sucrose gradients corresponding to the cultures infected with FBD/Poly-VP1 and FB/pVP2+FB/his-VP3, respectively. The total extracts (input) and the different fractions of the sucrose gradients (fraction F1 corresponds to the bottom of the gradient) were analyzed by means of Western blot using specific sera against the IBDV VP1, pVP2, VP3, and VP4 proteins, respectively. The molecular mass of the immunoreactive polypeptides is indicated in kDa.

FIG. 4. Biochemical and structural characterization of IBDV VLPs produced in S. cerevisiae transformed with the plasmid pESCURA/pVP2-VP3-GFP. A S. cerevisiae culture transformed with the plasmid pESCURA/pVP2-VP3-GFP was grown at 30° C. in a medium supplemented with the inducer galactose. At 18 hours, the culture was harvested and centrifuged. The resulting sediment was processed by means of fractioning in a 25-50% linear sucrose gradient. A) Biochemical analysis of samples corresponding to the sediment before fractioning (T) as well as the different fractions of the sucrose gradient. The samples were analyzed by means of SDS-PAGE and Western blot using specific antibodies against VP3 (anti-VP3) and pVP2 (anti-pVP2) proteins. The arrows indicate the positions of the immunoreactive bands corresponding to the VP3-GFP (61 kDa) and pVP2 (48 kDa) proteins, respectively. B) The structural analysis of the obtained samples was carried out by means of TEM. The image corresponds to a micrography obtained from an aliquot corresponding to the mixture of fractions 7, 8 and 9 of the sucrose gradient. The sample was stained with uranyl acetate and observed by means of TEM. The bar corresponds to 65 nm. C) VLPs sample obtained by means of the IBDV polyprotein expression in mammal cells by means of infection with the VT7/Poly recombinant vaccine virus (Fernández-Arias et al., (1998), cited supra). The bar corresponds to 65 nm.

### DETAILED DESCRIPTION OF THE INVENTION

In a first aspect, the invention provides an empty capsid of the infectious bursal disease virus (IBDV), hereinafter VLP(−VP4) of the invention, characterized in that it is constituted by assembly of only IBDV pVP2 proteins and IBDV VP3 proteins.

The term “IBDV”, as it is used in the present invention, refers to the different IBDV strains belonging to any of the known serotypes (1 or 2) [by way of illustration, see the review carried out by van den Berg T P, Eterradossi N, Toquin D, Meulemans G., en Rev Sci Tech 2000 19: 509-43] and the terms “IBDV pVP2 protein” and “IBDV VP3 protein” refer to the different forms of the pVP2 and VP3 proteins representative of any of the mentioned IBDV strains [NCBI protein databank], according to the definition made by Sánchez and Rodríguez (1999) (Sánchez A B, Rodríguez J F. Proteolytic processing in infectious bursal disease virus: identification of the polyprotein cleavage sites by site-directed mutagenesis. Virology. 1999 Sep. 15; 262(1):190-199), as well as proteins substantially homologous to said IBDV pVP2 and VP3 proteins, i.e. proteins the amino acid sequences of which have a degree of identity regarding said IBDV pVP2 and VP3 proteins of at least 60%, preferably of at least 80%, more preferably of at least 90% and even more preferably of at least 95%.

The IBDV pVP2 protein present in the VLP(−VP4) of the invention can be any pVP2 protein representative of any IBDV strain, for example, the full-length pVP2 protein of IBDV Soroa strain [NCBI, access number AAD30136]

The IBDV VP3 protein present in the VLP(−VP4) of the invention can be any VP3 protein representative of any IBDV strain, for example, the full-length VP3 protein of IBDV Soroa strain [NCBI, access number AAD30136].

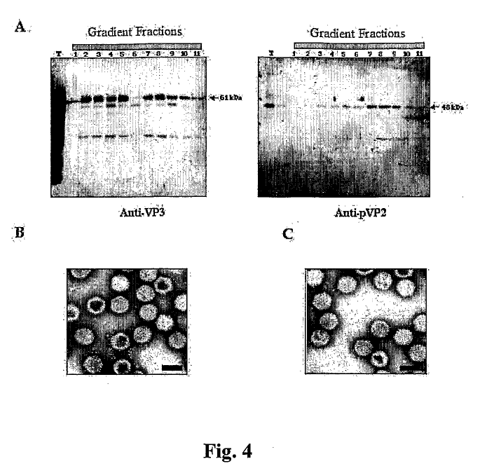

In a particular embodiment, the VLPs(−VP4) of the invention have a diameter of 65-70 nm and a polygonal contour indistinguishable from the IBDV VLPs obtained in other expression systems (FIG. 4C).

The VLPs(−VP4) of the invention can be obtained by means of the simultaneous expression of said IBDV pVP2 and VP3 proteins in suitable host cells. Said suitable host cells are cells containing the nucleotide sequence encoding the IBDV pVP2 protein and the nucleotide sequence encoding the IBDV VP3 protein, either in a single gene construct or in two gene constructs. In a particular embodiment, said suitable host cells are cells that are transformed, transfected or infected with a suitable expression system, such as an expression system comprising a gene construct, wherein said gene construct comprises the nucleotide sequence encoding for the IBDV pVP2 protein and the nucleotide sequence encoding the IBDV VP3 protein, or else alternatively with an expression system comprising a first gene construct comprising the nucleotide sequence encoding for the IBDV pVP2 protein, and a second gene construct comprising the nucleotide sequence encoding for the IBDV VP3 protein.

Therefore, in another aspect, the invention provides a nucleic acid, the nucleotide sequence of which comprises the nucleotide sequence encoding for said IBDV pVP2 protein and the nucleotide sequence encoding for said IBDV VP3 protein in the form of two independent genes. More specifically, the nucleic acid provided by this invention is characterized in that its nucleotide sequence is constituted by (i) a nucleotide sequence comprising the open reading frame corresponding to the IBDV pVP2 protein and (ii) a nucleotide sequence comprising the open reading frame corresponding to the IBDV VP3 protein. One feature of the nucleic acid provided by this invention is that it lacks the nucleotide sequence comprising the open reading frame corresponding to the IBDV VP4 protein. As it is used in this description, the term “open reading frame corresponding to the pVP2 protein” or “open reading frame corresponding to the IBDV VP3 proteins” includes, apart from the nucleotide sequences of said open reading frames, other open reading frames analogous to the same encoding frames of the IBDV pVP2 and VP3 proteins. As it is used herein, the term “analogous” intends to include any nucleotide sequence which can be isolated or constructed on the base of the encoding nucleotide sequence of IBDV pVP2 and VP3, for example by means of the introduction of conservative or non-conservative nucleotide replacements, including the insertion of one or more nucleotides, the addition of one or more nucleotides at any of the ends of the molecule, or the deletion of one or more nucleotides at any end or inside of the sequence. Generally, a nucleotide sequence analogous to another nucleotide sequence is substantially homologous to said nucleotide sequence. In the sense used in this description, the expression “substantially homologous” means that at the nucleotide level the nucleotide sequences in question have a degree of identity of at least 60%, preferably of at least 80%, more preferably of at least 90%, and even more preferably of at least 95%.

In another aspect, the invention provides a gene construct comprising a nucleic acid provided by this invention, i.e. a nucleic acid the nucleotide sequence of which is constituted by (i) a nucleotide sequence comprising the open reading frame corresponding to the IBDV pVP2 protein and (ii) a nucleotide sequence comprising the open reading frame corresponding to the IBDV VP3 protein. Said gene construct lacks the nucleotide sequence comprising the open reading frame corresponding to the IBDV VP4 protein.

In another aspect, the invention provides an expression vector or system selected from:
a) an expression system comprising a gene construct provided by this invention, operatively bound to transcription, and optionally translation, control elements, wherein said gene construct comprises the nucleotide sequence comprising the open reading frame corresponding to the IBDV pVP2 protein and the nucleotide sequence comprising the open reading frame corresponding to the IBDV VP3 protein; and
b) an expression system comprising two gene constructs, a first gene construct comprising the open reading frame corresponding to the IBDV pVP2 protein, operatively bound to transcription, and optionally translation, control elements, and a second gene construct comprising the open reading frame corresponding to the IBDV VP3 protein, operatively bound to transcription, and optionally translation, control elements.

In a particular embodiment, the expression system provided by this invention comprises a gene construct comprising (i) a nucleotide sequence comprising the open reading frame or encoding region corresponding to the IBDV pVP2 protein and (ii) a nucleotide sequence comprising the open reading frame or encoding region corresponding to the IBDV VP3 protein, wherein said gene construct is operatively bound to transcription, and optionally translation, control elements.

In another particular embodiment, the expression system provided by this invention comprises (i) a first gene construct, operatively bound to transcription, and optionally translation, control elements, said first gene construct comprising a nucleotide sequence comprising the open reading frame or encoding region corresponding to the IBDV pVP2 protein, and (ii) a second gene construct, operatively bound to transcription, and optionally translation, control elements, said second gene construct comprising a nucleotide sequence comprising the open reading frame or encoding region corresponding to the IBDV VP3 protein.

The transcription, and optionally translation, control elements present in the expression system provided by this invention include promoters, directing the transcription of the nucleotide sequences of interest to which they are operatively linked, and other sequences necessary or suitable for the transcription and its suitable regulation in time and place, for example, start and end signals, cleavage sites, polyadenylation signal, replication origin, transcriptional activators (enhancers), transcriptional silencers (silencers), etc.

Virtually any suitable expression system or vector can be used in the generation of the expression system provided by this invention. By way of illustration, said suitable expression or vector systems can be selected, according to the conditions and needs of each specific case, from plasmids, bacmids, yeast artificial chromosomes (YACs), bacteria artificial chromosomes (BACs), bacteriophage P1-based artificial chromosomes (PACs), cosmids, or viruses, which can further have a heterologous replication origin, for example, bacterial or of yeast, so that it may be amplified in bacteria or yeasts, as well as a marker usable for selecting the transfected cells different from the gene or genes of interest. These expression systems or vectors can be obtained by conventional methods known by persons skilled in the art [Sambrook, J., Fritsch, E. F., and Maniatis, T. (1989). Molecular cloning: a laboratory manual, 2nd ed. Cold Spring Harbor Laboratory] and form part of the present invention. In a particular embodiment, said expression or vector system is a plasmid, such as a plasmid suitable for transforming yeasts, for example, the plasmid called pESCURA/pVP2-VP3-GFP (Example 2), or a virus, such as a recombinant baculovirus (rBV), for example, the rBV called FBD/pVP2-his-VP3 (Example 1.2), simultaneously expressing both proteins (IBDV pVP2 and his-VP3) in insect cells during the replication cycle, or the rBVs called FB/pVP2 and FB/his-VP3 (Example 1.1) expressing the IBDV pVP2 and his-VP3 proteins, respectively, when coinfecting insect cells, IBDV VLPs(−VP4) being obtained.

In another aspect, the invention provides a host cell containing the encoding nucleotide sequence of the IBDV pVP2 protein and the encoding nucleotide sequence of the IBDV VP3 protein, either in a single gene construct or in two different gene constructs. In a particular embodiment, said host cell is a host cell that is transformed, transfected or infected with (i) an expression system provided by this invention comprising either a gene construct wherein said gene construct comprises the nucleotide sequence encoding for said IBDV pVP2 protein and the nucleotide sequence encoding for said IBDV VP3 protein, or else alternatively with (ii) an expression system comprising a gene construct comprising the nucleotide sequence encoding for said IBDV pVP2 protein and another gene construct comprising the nucleotide sequence encoding for said IBDV VP3 protein.

In a particular embodiment, the host cell provided by this invention is a host cell that is transformed, transfected or infected with an expression system comprising a gene construct comprising (i) a nucleotide sequence comprising the open reading frame or encoding region corresponding to the IBDV pVP2 protein and (ii) a nucleotide sequence comprising the open reading frame or encoding region corresponding to the IBDV VP3 protein, wherein said gene construct is operatively bound to transcription, and optionally translation, control elements.

In another particular embodiment, the host cell provided by this invention is a host cell that is transformed, transfected or infected with a first gene construct, operatively bound to transcription, and optionally translation, control elements, said first gene construct comprising a nucleotide sequence comprising the open reading frame or encoding region corresponding to the IBDV pVP2 protein, and with a second gene construct, operatively bound to transcription, and optionally translation, control elements, said second gene construct comprising a nucleotide sequence comprising the open reading frame or encoding region corresponding to the IBDV VP3 protein.

Virtually any host cell susceptible to being transformed, transfected or infected by an expression system provided by this invention can be used, for example, mammal cells, bird cells, insect cells, yeasts, etc; however, in a particular embodiment, said host cell is selected from yeasts and insect cells. Yeasts are suitable due to the simplicity and production cost. Insect cells are suitable when the expression system comprises one or two recombinant baculoviruses (rBV). The use of rBV is advantageous due to biosafety issues related to the host range of the baculoviruses, incapable of replicating in other cell types which are not insect cells.

In a particular embodiment, the invention provides a host cell, such as a yeast, for example, Saccharomyces cerevisiae, Saccharomyces pombe, etc., transformed with an expression system, such as a plasmid or an expression vector, comprising a gene construct provided by this invention comprising the nucleotide sequence encoding for said IBDV pVP2 protein and the nucleotide sequence encoding for the IBDV VP3 protein.

In another particular embodiment, the invention provides a host cell, such as an insect cell, infected with an expression system, such as a recombinant baculovirus, comprising a gene construct provided by this invention comprising the nucleotide sequence encoding for said IBDV pVP2 protein and the nucleotide sequence encoding for the IBDV VP3 protein.

In another particular embodiment, the invention provides a host cell, such as an insect cell, coinfected with an expression system comprising a first recombinant baculovirus comprising a gene construct provided by this invention comprising the nucleotide sequence encoding for said IBDV pVP2 protein and with a second recombinant baculovirus comprising a gene construct provided by this invention comprising the nucleotide sequence encoding for the IBDV VP3 protein.

In another aspect, the invention provides a process for the production of VLPs(−VP4) of the invention comprising culturing a host cell provided by this invention containing the encoding nucleotide sequence of said IBDV pVP2 protein and the encoding nucleotide sequence of said IBDV VP3 protein, either in a single gene construct or in two different gene constructs, and simultaneously expressing said IBDV pVP2 and VP3 proteins, and if so desired, recovering said VLPs(−VP4) of the invention. In a particular embodiment, said host cell provided by this invention is a cell that is transformed, transfected or infected with a suitable expression system, such as an expression system comprising a gene construct, wherein said gene construct comprises the nucleotide sequence encoding for said IBDV pVP2 protein and the nucleotide sequence encoding for said IBDV VP3 protein, or else alternatively with an expression system comprising a first gene construct comprising the nucleotide sequence encoding for said IBDV pVP2 protein and a second gene construct comprising the nucleotide sequence encoding for said IBDV VP3 protein.

Said process therefore comprises the gene coexpression of said IBDV pVP2 and VP3 proteins as two independent genes. After the simultaneous expression of said proteins (IBDV pVP2 and VP3) in said cells, the expressed proteins are assembled and form the VLPs(−VP4) of the invention, which can be isolated or withdrawn from the medium and purified if desired. The isolation and purification of said VLPs(−VP4) of the invention can be carried out by means of conventional methods, for example, by means of fractioning on sucrose gradients.

In a particular embodiment, the simultaneous gene coexpression of IBDV pVP2 and VP3 proteins is carried out by means of the use of an rBV allowing the simultaneous expression of said proteins from two independent chimeric genes in insect cells. In this case, the production of VLPs(−VP4) of the invention can be carried out by means of a process comprising, first, the obtainment of a gene expression system made up of an rBV containing a gene construct simultaneously encoding for said IBDV pVP2 and VP3 proteins, such as the rBV called FBD/pVP2-his-VP3 (Example 1.2), or alternatively the obtainment of an rBV containing a gene construct encoding for the IBDV pVP2 protein and the obtainment of another rBV containing a gene construct encoding for said IBDV VP3 protein, such as the rBVs called FB/pVP2 and FB/his-VP3 (Example 1.1), followed by the infection of insect cells with said expression system based on said recombinant baculovirus(es), expression of the recombinant proteins and if so desired, isolation of the VLPs(−VP4) of the invention formed by assembly of said IBDV pVP2 and VP3 proteins, and optionally subsequent purification of said VLPs(−VP4) of the invention.

The construction of a recombinant baculovirus allowing the independent expression of the IBDV pVP2 and VP3 proteins can be carried out by any person skilled in the art based on that described herein and on the state of the art concerning this technology (Cold Spring Harbor, N.Y.; Leusch M S, Lee S C, Olins P O. 1995. A novel host-vector system for direct selection of recombinant baculoviruses (bacmids) in Escherichia coli. Gene 160: 191-4; Luckow V A, Lee S C, Barry G F, Olins P O. 1993. Efficient generation of infectious recombinant baculoviruses by site-specific transposon-mediated insertion of foreign genes into a baculovirus genome propagated in Escherichia coli. J Virol 67: 4566-79).

In another particular embodiment, the gene coexpression of the IBDV pVP2 and VP3 proteins is carried out by means of a vector allowing the expression of said proteins in yeast cells. In this case, the production of VLPs(−VP4) of the invention can be carried out by means of a process comprising, first, the obtainment of a gene expression system made up of a plasmid containing a gene construct simultaneously encoding for the IBDV pVP2 and VP3 proteins, followed by the transformation of yeasts with said expression system, expression of the recombinant proteins, and if so desired, isolation of the VLPs(−VP4) of the invention formed by assembly of said IBDV pVP2 and VP3 proteins, and optionally subsequent purification of said VLPs(−VP4) of the invention. In a specific embodiment, the suitable expression system for transforming yeasts is based on a pESC Yeast (Stratagene) expression system such as, for example, the plasmid pESCURA/pVP2/VP3-GFP (Example 2) containing a gene construct encoding for the IBDV pVP2 and VP3-GFP proteins.

The obtainment of yeasts transformed with a gene construct or with a suitable expression system or vector allowing the simultaneous expression of the IBDV pVP2 and VP3 proteins can be carried out by any person skilled in the art based on that described herein and on the state of the art concerning this technology (pESC epitope tagging vectors Instructions manual. Stratagene www.stratagene.com; Sambrook, J., Fritsch, E. F., and Maniatis, T. (1989). Molecular cloning: a laboratory manual, 2nd ed. Cold Spring Harbor Laboratory).

In another aspect, the invention is related to the use of the gene expression system provided by this invention for producing and obtaining the VLPs(−VP4) of the invention.

The VLPs(−VP4) of the invention can be used to immunize animals, particularly birds, per se or as vectors or vehicles of molecules with biological activity, for example, polypeptides, proteins, nucleic acids, drugs, etc., whereby they can be used for therapeutic or diagnostic purposes. In a particular embodiment, said molecules with biological activity include antigens or immune response inducers in animals or humans to whom they are supplied, or drugs which can be released in their specific action site, or nucleic acid sequences, all being useful in gene therapy and intended for being introduced inside the suitable cells.

Therefore, in another aspect, the invention is related to the use of the VLPs(−VP4) of the invention in the manufacture of medicaments such as vaccines, gene therapy vectors (delivery systems), etc. In a particular embodiment, said medicament is a vaccine intended for conferring protection to animals, particularly birds, against the infectious bursal disease virus (IBDV). In another particular embodiment, said medicament is a gene therapy vector.

In another aspect, the invention provides a vaccine comprising a therapeutically effective amount of VLPs(−VP4) of the invention, optionally together with one or more pharmaceutically acceptable adjuvants and/or vehicles. Said vaccine is useful for protecting animals, particularly birds, against the infectious bursal disease virus (IBDV). In a particular embodiment, said birds are selected from the group formed by chickens, turkeys, geese, ganders, pheasants, quails and ostriches. In a preferred embodiment, the vaccine provided by this invention is a vaccine useful for protecting chickens from the infection caused by IBDV.

In the sense used in this description, the expression “therapeutically effective amount” refers to the amount of VLPs(−VP4) of the invention calculated for producing the desired effect and will generally be determined, among others, by the characteristics of the VLPs(−VP4) and the immunization effect to be achieved.

The pharmaceutically acceptable adjuvants and vehicles which can be used in said vaccines are those adjuvants and vehicles known by the persons skilled in the art and normally used in the manufacture of vaccines.

In a particular embodiment, said vaccine is prepared in form of an aqueous solution or suspension in a pharmaceutically acceptable diluent, such as saline solution, phosphate-buffered saline solution (PBS), or any other pharmaceutically acceptable diluent.

The vaccine provided by this invention can be administered by any suitable administration route which results in a protective immune response against the heterologous sequence or epitope used, to which end said vaccine will be formulated in the dosage form suited to the chosen administration route. In a particular embodiment, the administration of the vaccine provided by this invention is carried out parenterally, for example, intraperitoneally, subcutaneously, etc.

The following Examples illustrate the invention and should not be considered limiting of the scope thereof.

### EXAMPLE 1

### Obtaining VLPs(−VP4) by Means of the Coexpression of pVP2 (pVPX) and VP3 in Insect Cells

1.1 Obtaining VLPs(−VP4) by Means of the Coexpression of pVP2 (pVPX) and VP3 with Two Independent rBVs in Insect Cells

The results of a series of experiments designed to analyze the possibility of obtaining IBDV VLPs from a strategy avoiding the synthesis of the IBDV polyprotein and, therefore, the presence of the VP4 protease during the assembly process are described in this example. The experimental design is based on the coexpression of the IBDV pVP2 and VP3 proteins from two independent chimeric genes. To that end, two recombinant baculoviruses (rBVs) described above, FB/his-VP3 (Kochan, G., González, D. & Rodríguez, J. F. (2003). Characterization of the RNA binding activity of VP3, a major structural protein of IBDV. Archives of Virology 148, 723-744) and FB/VPX [also identified as FB/pVP2 in this description] (Martínez-Torrecuadrada, J. L., Castón, J. R., Castro, M., Carrascosa, J. L., Rodríguez, J. F. & Casal, J. I. (2000). Different architectures in the assembly of infectious bursal disease virus capsid proteins expressed in insect cells. Virology 278, 322-331) have been used. These rBVs were generated by means of the cloning into suitable vectors of the complementary DNA (cDNA) encoders of the IBDV pVP2 and pVP3 proteins. Said cDNAs were obtained by RT-PCR from the A segment of the serotype I IBDV Soroa strain genome a (NCBI access number AAD30136). The rBV FB/his-VP3 expresses a chimeric VP3 protein which at its N-terminal end contains a tandem of six histidines fused to the VP3 sequence (Met754-Glu1012 of the polyprotein) called his-VP3. rBV FB/pVP2 expresses the encoding region of the pVP2 protein (Met1-Ala512).

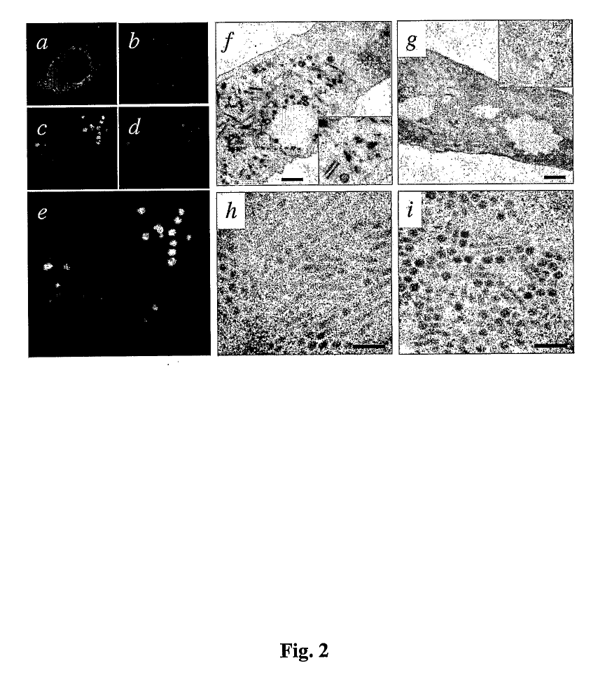

The analysis of the expression of these pVP2 and his-pVP3 proteins, whether independently or together, was carried out in cell cultures. To carry out these experiments, single layer cell cultures from the insect Trichloplusia ni (H5, Invitrogen) were used, which were grown on cover glasses. Said cultures were independently infected with FB/pVP2, FB/his-VP3, or coinfected with both rBVs. The multiplicity of infection was 5 plaque forming units (pfu)/cell. The cells were fixed at 48 hours post-infection (h.p.i), and incubated with rabbit anti-VP2 polyclonal serum and with rat anti-VP3 polyclonal serum (Fernández-Arias, A., Risco, C., Martínez, S., Albar, J. P. & Rodríguez, J. F. (1998). Expression of ORF A1 of infectious bursal disease virus results in the formation of virus-like particles. Journal of General Virology 79, 1047-1054). After successive washings, the cover glasses were incubated with goat anti-rat serum conjugated with Alexa 594 and with goat anti-rabbit serum conjugated with Alexa 488 (Jackson Immunoresearch Laboratories, Inc.). The cellular cores were stained with the specific To-Pro-3 marker (Molecular Probes, Inc.). The samples were finally viewed by epifluorescence with a Zeiss Axiovert 200 microscope equipped with the Bio Rad Radiance 2100 confocal system. The images obtained were stored using the Laser Sharp Package (Bio Rad) software equipment. As is shown in FIG. 2a, in the cultures infected with FB/pVP2, the anti-VP2 serum showed a fine granular signal mixed with tubular structures, both distributed throughout the cytoplasm. The anti-VP3 signal, detected in the cells infected with rBV FB/his-VP3, was characterized by the presence of spherical-shaped, and apparently hollow, accumulations around the core. In the cultures coinfected with both rBVs, a notable modification in the distribution pattern of both proteins was detected. In these cells, the specific signals of pVP2 and VP3 were collocated in spherical and dense accumulations, suggesting that their coexpression allowed the formation of pVP2/his-VP3 complexes (FIG. 2c to 2e).

For the purpose of characterizing these structures in greater detail, similar extracts corresponding to cells infected with FB/pVP2+FB/hisVP3 were analyzed by transmission electron microscopy (TEM). As a control, and in parallel, H5 cell cultures infected with the wild strain of the FBD (FastBacDual, Invitrogen) virus were analyzed by the same technique. After the infection, the cells were harvested after 48 hours, and processed as has been previously described (Fernández-Arias A, Risco C, Martínez S, Albar J P & Rodríguez J F. (1998). Expression of ORF A1 of infectious bursal disease virus results in the formation of virus-like particles. Journal of General Virology 79:1047-1054) for their analysis in ultrathin sections by TRANSMISSION ELECTRON MICROSCOPY. As is shown in FIG. 2, the cytoplasm of the coinfected cells contains aggregates formed by a mixture of tubules and structures similar to capsids (FIGS. 2g, 2h and 2i). These aggregates were not observed in any case in the samples corresponding to cells infected with wild FBD virus (FIG. 2f). The appearance and size of the tubules, as well as of the structures similar to capsids, was similar to those previously described in cell cultures infected with VT7/Poly, a recombinant of the vaccinia virus expressing the gene of the IBDV polyprotein (Fernández-Arias A, Risco C, Martínez S, Albar J P & Rodríguez J F. (1998). Expression of ORF A1 of infectious bursal disease virus results in the formation of virus-like particles. Journal of General Virology 79:1047-1054).

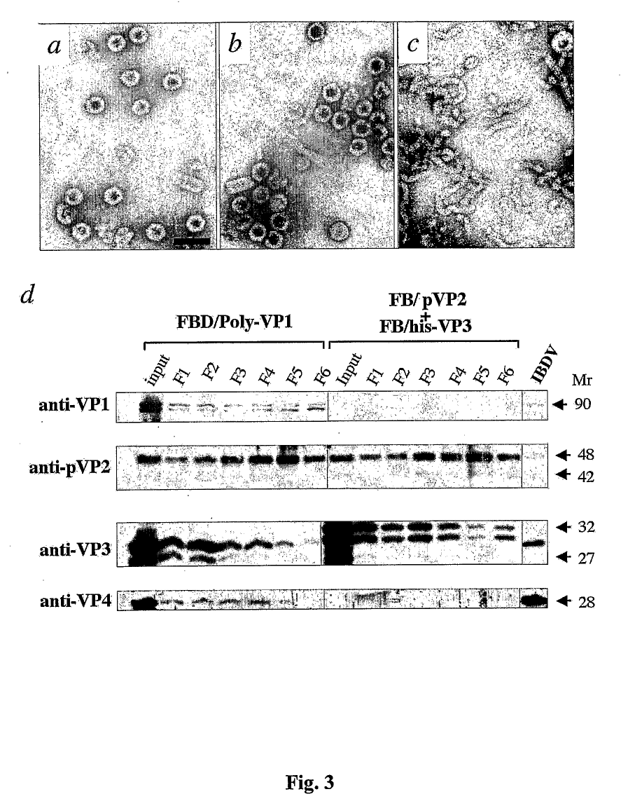

To unmistakably establish that the coexpression of pVP2 and his-VP3 enabled the assembly and, therefore, the obtainment of VLPs-(VP4), the decision was made to purify the formed particles. To that end, H5 cell cultures were infected with FB/pVP2+FB/his-VP3. At 60 h.p.i., the cells were homogenized and the extracts were separated on sucrose gradients as previously described (Lombardo E, Maraver A, Castón J R, Rivera J, Fernández-Arias A, Serrano A, Carrascosa J L & Rodríguez J F. (1999). VP1, the putative RNA-dependent RNA polymerase of infectious bursal disease virus, forms complexes with the capsid protein VP3, leading to efficient encapsidation into virus-like particles. Journal of Virology 73:6973-6983). After their centrifugation, the gradients were fractioned, and the different fractions were analyzed by TEM as previously described (Lombardo and et al., cited supra). As a control, and subject to the same process, gradients corresponding to cell extracts infected with rBV FB/VPX or with rBV FBD/Poly-VP1, were fractioned. The recombinant virus FBD/Poly-VP1 simultaneously expresses the VP1 polypeptide and polyprotein. As was predictable, the infection with FBD/Poly-VP1 had a result of an efficient production of VLPs (Maraver A, Oña A, Abaitua F, González D, Clemente R, Diaz-Ruiz A, Castón J R, Pazos F & Rodríguez J F. (2003). The oligomerization domain of VP3, the scaffolding protein of infectious bursal disease virus, plays a critical role for capsid formation. Journal of Virology 77:6438-49). On the other hand, the fractions corresponding to the cells infected with FB/VPX only contain tubules of a twisted appearance. The gradients corresponding to cells coinfected with the rBVs FB/pVP2+FB/his-VP3 contain rigid type I tubules in the fractions near the bottom of the gradient, and VLPs-(VP4) in the central and top fractions (FIG. 3b). The VLPs-(VP4) isolated from the cells coinfected with rBV FB/pVP2+FB/his-VP3 had a diameter of 65-70 nm, as well as a typical polygonal contour, absolutely indistinguishable from the purified VLPs of cultures infected with FBD/Poly-VP1 (Maraver, A., Oña, A., Abaitua, F., González, D., Clemente, R., Diaz-Ruiz, A., Caston, J. R., Pazos, F. & Rodríguez, J. F. (2003). The oligomerization domain of VP3, the scaffolding protein of infectious bursal disease virus, plays a critical role for capsid formation. Journal of Virology 77:6438-49) or of the cultures infected with VT7/Poly (Fernández-Arias, A., Risco, C., Martínez, S., Albar, J. P. & Rodríguez, J. F. (1998). Expression of ORF A1 of infectious bursal disease virus results in the formation of virus-like particles. Journal of General Virology 79, 1047-1054).

For the purpose of achieving a biochemical characterization of the obtained material, Western blot experiments were carried out in which the different fractions were compared with specific sera against the VP1, pVP2, VP3 and VP4 proteins (Fernández-Arias et al. 1998, Lombardo et al., 2000). Cell extracts infected with IBDV were used as a control. The obtained results are shown in FIG. 3d. As was expected, the bands corresponding to the VP1 and VP4 polypeptides were only detected in samples corresponding to cells infected with FBD/Poly-VP1. The patterns corresponding to pVP2/VP3 in samples corresponding to cells infected with FBD/Poly-VP1 or coinfected with FB/VPX+FB/his-VP3 were similar, two bands corresponding to pVP2 and VP3, respectively, being detected.

1.2 Obtaining VLPs(−VP4) by Means of the Coexpression of pVP2 (pVPX) and VP3 with an rBV in Insect Cells

Furthermore, the construction of the plasmid pFBD/pVP2-his-VP3 was carried out. The first step of the construction was carried out by means of the cloning of the encoding region of the pVP2 protein into the pFBDual vector (Invitrogen). The DNA fragment corresponding to pVP2 was obtained by means of PCR with the oligonucleotides identified as Oligo I (SEQ ID NO: 1) and Oligo II (SEQ ID NO: 2) using the plasmid pVOTE.2/Poly as a mold (Fernández-Arias, A., Risco, C., Martínez, S., Albar, J. P. & Rodríguez, J. F. (1998). Expression of ORF A1 of infectious bursal disease virus infectious results in the formation of virus-like particles. Journal of General Virology 79, 1047-1054). The fragment was purified, subjected to digestion with the BglII and HindIII enzymes and cloned into the pFBDual vector (Invitrogen) previously digested with the BamHI and Hindi enzymes. The resulting plasmid was called pFBD/pVP2. Then, a DNA fragment containing the open reading frame corresponding to the VP3 protein was obtained by means of digestion of the plasmid pFB/his-VP3 (Kochan et al., 2003, cited supra) with the RsrII enzyme, treatment with Klenow, and subsequent restriction with KpnI. This DNA fragment was purified and cloned into the plasmid-pFBD/pVP2 previously digested with the SmaI and KpnI enzymes. The resulting plasmid was called pFBD/pVP2-his-VP3 (SEQ ID NO: 3) and contains the encoding nucleotide sequence of the pVP2 and his-pVP3 proteins (the latter is encoded by the complementary chain to the nucleotides 6734-7585 of SEQ ID NO: 3). The amino acid sequence of the pVP2 protein and of the his-VP3 fusion protein (pVP2-his-VP3) encoded by the nucleotide sequence contained in said plasmid pFBD/pVP2-his-VP3 is shown in SEQ ID NO: 4.

The plasmid pFBD/pVP2-his-VP3 allows obtaining an rBV, called FBD/pVP2-his-VP3, expressing both proteins simultaneously during its replication cycle [http://invitrogen.com/content/sfs/manuals/bevtest.pdf].

The results obtained with FBD/pVP2-his-VP3 are identical to those obtained by means of the coinfection with rBVs FB/pVP2 and FD/his-VP3, IBDV VLPs(−VP4) being obtained.

### EXAMPLE 2

### Obtaining VLPs(−VP4) by Means of the Coexpression of pVP2 and VP3 as Two Independent Genes in Yeasts

For the purpose of studying the possibility of obtaining IBDV VLPs(−VP4) in yeast cultures (Saccharomyces cerevisiae) the vector pESCURA/pVP2-VP3-GFP was generated with the heterologous GFP gene bound to the VP3 N-terminal end. The first step in the construction of the vector was carried out by means of the cloning of the encoding region of the IBDV pVP2 protein into the vector pESCURAinv. The plasmid pESCURAinv was generated by means of digestion of the vector pRS426 (Stratagene) with the PvuII enzyme and religation of the digestion mixture. The resulting vector, pESCURAinv, contains the multiple cloning region in reversed position with regard to that of parent vector pRS426. The DNA fragment corresponding to the pVP2 protein was obtained by means of PCR with the oligonucleotides called Oligo III (SEQ ID NO: 5) and Oligo IV (SEQ ID NO: 6) using the plasmid pVOTE.2/Poly as a mold (Fernández-Arias, A., Risco, C., Martínez, S., Albar, J. P. & Rodríguez, J. F. (1998). Expression of ORF A1 of infectious bursal disease virus results in the formation of virus-like particles. Journal of General Virology 79, 1047-1054). The fragment was purified subjected to digestion with the BglII and HindIII enzymes and cloned into the vector pESCURA.inv, previously digested with the BamHI and HindIII enzymes. The resulting plasmid was called pESCURA/pVP2.

The plasmid pFB/VP3-GFP was constructed in two stages. The first one consisted of the cloning of a DNA fragment, generated by means of PCR, containing the ORF of the IBDV VP3 protein lacking the termination codon. This PCR was carried out using the oligonucleotides called Oligo V (SEQ ID NO: 7) and Oligo VI (SEQ ID NO: 8) and using the plasmid pVOTE.2/Poly as a mold (Fernández-Arias, A., Risco, C., Martínez, S., Albar, J. P. & Rodríguez, J. F. (1998). Expression of ORF A1 of infectious bursal disease virus results in the formation of virus-like particles. Journal of General Virology 79, 1047-1054). The resulting DNA was digested with the EcoRI and BamHI enzymes and cloned into the vector pEGFP-N3 (Clontech), also digested with the same enzymes. The resulting plasmid was called pVP3-GFP. Then, the plasmid pEGFP-GFP was digested with the EcoRI and NotI enzymes and cloned into the vector pFastBac1 (Invitrogen). The resulting plasmid was called pFB/VP3-GFP.

Then, a DNA fragment that contained the open reading frame corresponding to the IBDV VP3 protein fused to the encoding region of the EGFP protein was obtained by means of digestion of the plasmid pFB/VP3-GFP with the EcoRI and NotI enzymes. This DNA fragment was purified and cloned into the plasmid pESCURA/pVP2 previously digested with the EcoRI and NotI enzymes. The resulting plasmid was called pESCURA/pVP2-VP3-GFP (SEQ ID NO: 9) and contains the ORFs of the pVP2 and VP3-GFP proteins under the transcriptional control of two independent promoters, GAL 1 and GAL 10, both inducible by galactose (the pVP2 protein is encoded by the chain of nucleotides complementary to the nucleotides 5862-7343 of SEQ ID NO: 9). The amino acid sequence of the pVP2 protein and of the VP3-GFP fusion protein (pVP2-VP3-GFP) encoded by the nucleotide sequence contained in said plasmid pESCURA/pVP2-VP3-GFP is shown in SEQ ID NO: 10.

pESCURA/pVP2-VP3-GFP was subsequently used to transform a culture of S. cerevisiae yeast haploid strain 499 according to a previously described protocol (Gietz, R. D. and R. A. Woods. (2002), Transformation of yeast by the Liac/SS carrier DNA/PEG method. Methods in Enzymology 350:87-96). The yeasts transformed with the plasmid were selected by means of growth on SC medium plates (CSM+YND, 2% glucose and bacto agar) supplemented with the amino acids tryptophan, leucine and histidine and lacking uracyl (−Ura). After an incubation of 48 hours at 30° C., a colony was chosen which was used to carry out the following protein expression and VLPs-(VP4) formation analyses.

The pVP2 and VP3 protein expression and VLPs-(VP4) formation analyses were carried out following a protocol previously described for the characterization of IBDV VLPs in other expression systems (Fernández-Arias, A., Risco, C., Martínez, S., Albar, J. P. & Rodríguez, J. F. (1998). Expression of ORF A1 of infectious bursal disease virus results in the formation of virus-like particles. Journal of General Virology 79, 1047-1054; Lombardo, E., Maraver, A., Castón, J. R., Rivera, J., Fernández-Arias, A., Serrano, A., Carrascosa, J. L. & Rodríguez, J. F. (1999). VP1, the putative RNA-dependent RNA polymerase of infectious bursal disease virus, forms complexes with the capsid protein VP3, leading to efficient encapsidation into virus-like particles. Journal of Virology 73, 6973-698). The colony selected was cultured in liquid CSM (−Ura)+YNB medium supplemented with 2% raffinose. The culture was incubated at 30° C. for 24 hours. This culture was used to inoculate, at an optical density (O.D.) of 0.2, a flask of 200 ml of CSM (−Ura)+YNB medium supplemented with 2% inducer galactose. The culture was maintained at 30° C. for 18 hours (until an O.D. between 1.0 and 2.0). The yeasts were centrifuged at 3,000 radiant power measurement, 5 minutes at 4° C., were washed once with distilled water, and the pellet was resuspended in lysis buffer (TEN: Tris 10 mM, pH 8.0; NaCl 150 mM; EDTA 1 mM)+2×protease inhibitors (Compl Roche). A volume of glass beads having a size of about 425-600 microns (Sigma) were added for the lysis. This mixture was subjected to vigorous vortex stirring for 30 seconds 4 times, with 30-second intervals, and at 4° C. After this, the soluble fraction was recovered by centrifuging the lysis mixture at 13,000 rpm for 15 minutes at 4° C. This sample was subjected to fractioning on a sucrose gradient according to a previously described protocol (Lombardo, E., Maraver, A., Castón, J. R., Rivera, J., Fernández-Arias, A., Serrano, A., Carrascosa, J. L. & Rodríguez, J. F. (1999). VP1, the putative RNA-dependent RNA polymerase of infectious bursal disease virus, forms complexes with the capsid protein VP3, leading to efficient encapsidation into virus-like particles. Journal of Virology 73, 6973-6983). The samples obtained after fractioning as well as a sample of the starting material were analyzed by means of sodium dodecylsulfate polyacrylamide gel electrophoresis (SDS-PAGE) [Current Protocols in Molecular Biology] and immunodetection by Western blot (FIG. 4A) using anti-pVP2 and anti-VP3 sera [Current Protocols in Molecular Biology]. As is shown in FIG. 4A, the Western blot showed the presence of bands, with the predicted molecular mass corresponding to the pVP2 (48 kDa) and VP3-GFP (61 kDa) proteins, as well as other immunoreactive bands of a smaller size probably produced by proteolytic degradation both in the initial sample and in the different fractions of the gradient. These results reliably showed the correct expression of both polypeptides in the S. cerevisiae culture transformed with the plasmid pESCURA/pVP2-VP3. Then, the different fractions of the gradient were analyzed by means of TEM as has been previously described (Lombardo, E., Maraver, A., Castón, J. R., Rivera, J., Fernández-Arias, A., Serrano, A., Carrascosa, J. L. & Rodríguez, J. F. (1999). VP1, the putative RNA-dependent RNA polymerase of infectious bursal disease virus, forms complexes with the capsid protein VP3, leading to efficient encapsidation into virus-like particles. Journal of Virology 73, 6973-6983). As is shown in FIG. 4B, the TEM analysis of the fractions of the gradient showed the existence of IBDV VLPs in the top fractions of the gradient. These VLPs, VLPs(−VP4), have a diameter of 65-70 nm and a polygonal contour that is indistinguishable from the IBDV VLPs obtained in other expression systems (FIG. 4C).

1. An empty capsid of the infectious bursal disease virus (IBDV) consisting of a plurality of IBDV pVP2 proteins and IBDV VP3 proteins.

2. A nucleic acid comprising (i) a nucleotide sequence comprising an open reading frame encoding an IBDV pVP2 protein and (ii) a nucleotide sequence comprising an open reading frame encoding an IBDV VP3 protein.

3. A vector gene comprising the nucleic acid according to claim 2.

4. An expression system comprising:
(i) a first polynucleotide sequence comprising an open reading frame encoding an IBDV pVP2 protein, and (ii) a second polynucleotide sequence comprising an open reading frame encoding an IBDV VP3 protein,
wherein the first and second polynucleotide sequences are operatively bound to at least one transcription control element.

5. An expression system according to claim 4, wherein said expression system comprises one or more plasmids, bacmids, yeast artificial chromosomes (YACs), bacteria artificial chromosomes (BACs), bacteriphage P1-based artificial chromosomes (PACs), cosmids, and/or viruses.

6. A host cell comprising one or more nucleic acids, which one or more nucleic acids comprise (i) a nucleotide sequence comprising an open reading frame encoding an IBDV pVP2 protein and (ii) a nucleotide sequence comprising an open reading frame encoding an IBDV VP3 protein.

7. The host cell of claim 6, wherein the host cell is transformed, transfected or infected with an expression system comprising the one or more nucleic acids.

8. The host cell according to claims 6, wherein the host cell is an insect cell or a yeast cell.

9. A method for producing the empty capsid of claim 1, comprising culturing a host cell comprising one or more nucleic acids, which one or more nucleic acids comprise (i) a nucleotide sequence comprising an open reading frame encoding an IBDV pVP2 protein and (ii) a nucleotide sequence comprising an open reading frame encoding an IBDV VP3 protein.

10. The method according to claim 9, comprising:
a) infecting insect cells with one or more recombinant baculoviruses, which one or more recombinant baculovirus comprise (i) a nucleotide sequence comprising an open reading frame encoding an IBDV pVP2 protein and (ii) a nucleotide sequence comprising an open reading frame encoding an IBDV VP3 protein; and
b) culturing the infected insect cells obtained in step a) under conditions allowing the expression of recombinant proteins and their assembly into empty IBDV capsids.

11. The method according to claim 9, comprising:
a) transforming yeast cells with one or more plasmids, which one or more plasmids comprise (i) a nucleotide sequence comprising an open reading frame encoding an IBDV pVP2 protein and (ii) a nucleotide sequence comprising an open reading frame encoding an IBDV VP3 protein; and
b) culturing the transformed yeast cells obtained in step b) under conditions allowing the expression of recombinant proteins and their assembly into empty IBDV capsids.

12. (canceled)

13. A pharmaceutical composition comprising an empty capsid of the infectious bursal disease virus (IBDV) according to claim 1.

14. The pharmaceutical composition according to claim 13, wherein said pharmaceutical composition is a vaccine against avian infectious bursal disease.

15. The pharmaceutical composition according to claim 13, wherein said pharmaceutical composition is a gene therapy vector.

16. A vaccine comprising a therapeutically effective amount of the empty IBDV capsids, according to claim 1.

17. The vaccine according to claim 16, wherein the vaccine is capable of eliciting an immune response capable of protecting birds from an infection caused by the infectious bursal disease virus (IBDV).

18. The vaccine according to claim 17, wherein said birds are selected from the group consisting of chickens, turkeys, geese, ganders, pheasants, quails, and ostriches.

19. (canceled)

20. The expression system of claim 4, wherein the first and second polynucleotide sequences are on the same or different nucleic acids.

21. The expression system of claim 5, wherein the expression system further comprises a heterologous replication origin.

22. The method according to claim 9, further comprising recovering said empty capsid.

23. The method according to claim 10, further comprising recovering said empty capsids.

24. The method according to claim 11, further comprising recovering said empty capsids.

25. The vaccine according to claim 16, further comprising at least one pharmaceutically acceptable vehicle and/or adjuvant.
